(19) United States
(12) Patent Application Publication
(10) Pub. No.: US 2026/0090891 A1
(43) Pub. Date: Apr. 2, 2026

(54) SYSTEMS AND METHODS FOR MULTI-PLANAR ORTHOPEDIC ALIGNMENT

(71) Applicant: Carlsmed, Inc., Carlsbad, CA (US)

(72) Inventor: Michael J. CORDONNIER, Carlsbad, CA (US)

(21) Appl. No.: 19/321,091

(22) Filed: Sep. 5, 2025

Related U.S. Application Data
(63) Continuation of application No. 17/369,777, filed on Jul. 7, 2021, now Pat. No. 12,427,025.
Application No. 17/369,777 is a continuation of application No. 16/207,116, filed on Dec. 1, 2018, now Pat. No. 11,083,586.
(60) Provisional application No. 62/594,510, filed on Dec. 4, 2017.

(51) Int. Cl. A61F 2/30; A61F 2/44

(52) U.S. Cl. CPC A61F 2/30942; A61F 2/4455; A61F 2002/30593; A61F 2002/30943; A61F 2002/30948; A61F 2002/30952; A61F 2002/30953; A61F 2002/30985; A61F 2/446; A61F 2/4465; A61F 2/447

(57) ABSTRACT

A method for designing a patient-specific implant includes obtaining image data of a region of interest of the spine of a patient, measuring one or more geometric characteristic of the region of interest from the image data, comparing a measurement obtained for at least one of the one or more geometric characteristics to a mathematical rule associated with the particular geometric characteristic, and generating three-dimensional implant geometry data if the measurement of the at least one of the one or more geometric characteristics conforms with the associated mathematical rule, the implant geometry data configured to guide an additive manufacturing operation.

### CROSS-REFERENCE TO RELATED APPLICATIONS

This application is a continuation of U.S. patent application Ser. No. 17/369,777, filed on Jul. 7, 2021, which is a continuation of U.S. patent application Ser. No. 16/207,116, filed on Dec. 1, 2018, now U.S. Pat. No. 11,083,586 issued on Aug. 10, 2021, which claims priority to U.S. Provisional Ser. No. 62/594,510 , filed on Dec. 4, 2017, each of which are incorporated herein by reference in their entireties for all purposes.

### TECHNICAL FIELD

The field of the invention generally relates to patient-specific implants, including patient-specific orthopedic implants, and methods for designing and producing them.

### BACKGROUND

Orthopedic implants are used to correct a variety of different maladies. Orthopedic surgery utilizing orthopedic implants may include one of a number of specialties, including: hand surgery, shoulder and elbow surgery, total joint reconstruction (arthroplasty), skull reconstruction, pediatric orthopedics, foot and ankle surgery, spine surgery, musculoskeletal oncology, surgical sports medicine, and orthopedic trauma. Spine surgery may encompass one or more of the cervical, thoracic, lumbar spine, or the sacrum, and may treat a deformity or degeneration of the spine, or related back pain, leg pain, or other body pain. Irregular spinal curvature may include scoliosis, lordosis, or kyphosis (hyper or hypo), and irregular spinal displacement may include spondylolisthesis. Other spinal disorders include osteoarthritis, lumbar degenerative disc disease or cervical degenerative disc disease, lumbar spinal stenosis or cervical spinal stenosis.

Spinal fusion surgery may be performed to set and hold purposeful changes imparted on the spine. Spinal fusion procedures include PLIF (posterior lumbar interbody fusion), ALIF (anterior lumbar interbody fusion), TLIF (transverse or transforaminal lumbar interbody fusion), or LLIF (lateral lumbar interbody fusion), including DLIF (direct lateral lumbar interbody fusion) or XLIF (extreme lateral lumbar interbody fusion).

One goal of interbody fusion is to grow bone between vertebra in order to seize (e.g., lock) the spatial relationships in a position that provides enough room for neural elements, including exiting nerve roots. An interbody implant device (interbody device, interbody implant, interbody cage, fusion cage, or spine cage) is a prosthesis used in spinal fusion procedures to maintain relative position of vertebra and establish appropriate foraminal height and decompression of exiting nerves. Each patient may have individual or unique disease characteristics, but most implant solutions include implants (e.g., interbody implants) having standard sizes or shapes (stock implants).

### SUMMARY

In one embodiment of the present disclosure, a method for designing a patient-specific implant includes obtaining image data of a region of interest of the spine of a patient, measuring one or more geometric characteristic of the region of interest from the image data, comparing a measurement obtained for at least one of the one or more geometric characteristics to a mathematical rule associated with the particular geometric characteristic, and generating three-dimensional implant geometry data if the measurement of the at least one of the one or more geometric characteristics conforms with the associated mathematical rule, the implant geometry data configured to guide an additive manufacturing operation.

In another embodiment of the present disclosure, a method for designing a patient-specific implant includes obtaining image data of a region of interest of the spine of a patient, measuring one or more geometric characteristic of the region of interest from the image data, comparing a measurement obtained for at least one of the one or more geometric characteristics to a mathematical rule associated with the particular geometric characteristic, and generating three-dimensional implant geometry data if the measurement of the at least one of the one or more geometric characteristics conforms with the associated mathematical rule, the implant geometry data configured to guide a subtractive manufacturing operation.

In still another embodiment of the present disclosure, a method for designing a patient-specific implant includes obtaining image data of a region of interest of the spine of a patient, measuring one or more geometric characteristic of the region of interest from the image data, comparing a measurement obtained for each member of a selected group of the one or more geometric characteristics to a mathematical rule associated with the particular geometric characteristic, creating a corrected value for any of the one or more geometric characteristics that do not conform with the associated mathematical rule, and generating three-dimensional implant geometry data configured to guide an additive manufacturing operation.

In yet another embodiment of the present disclosure, a method for designing a patient-specific implant includes obtaining image data of a region of interest of the spine of a patient, measuring one or more geometric characteristic of the region of interest from the image data, comparing a measurement obtained for each member of a selected group of the one or more geometric characteristics to a mathematical rule associated with the particular geometric characteristic, creating a corrected value for any of the one or more geometric characteristics that do not conform with the associated mathematical rule, and generating three-dimensional implant geometry data configured to guide a subtractive manufacturing operation.

In still another embodiment of the present disclosure, a method for designing a patient-specific implant includes obtaining or uploading computed tomography (CT) data from a spine of a subject, converting the computed tomography (CT) data into a three-dimensional image, selecting or indicating selected segments of the spine for applying surgical correction, applying one or more correction guidelines for the selected segments of the spine; determining whether the selected segments of the spine conform to the one or more correction guidelines, and generating three-dimensional implant geometry data configured to guide an automated manufacturing operation configured to produce an implant.

In yet another embodiment of the present disclosure, a computer system for designing a patient-specific implant includes at least one computer memory that is not a transitory signal, the at least one computer memory including instructions executable by at least one processor for obtaining image data of a region of interest of the spine of a patient, measuring one or more geometric characteristic of the region of interest from the image data, comparing a measurement obtained for at least one of the one or more geometric characteristics to a mathematical rule associated with the particular geometric characteristic, and generating three-dimensional implant geometry data if the measurement of the at least one of the one or more geometric characteristics conforms with the associated mathematical rule, the implant geometry data configured to guide an additive manufacturing operation.

In still another embodiment of the present disclosure, a computer system for designing a patient-specific implant includes at least one computer memory that is not a transitory signal, the at least one computer memory including instructions executable by at least one processor for obtaining image data of a region of interest of the spine of a patient, measuring one or more geometric characteristic of the region of interest from the image data, comparing a measurement obtained for each member of a selected group of the one or more geometric characteristics to a mathematical rule associated with the particular geometric characteristic, creating a corrected value for any of the one or more geometric characteristics that do not conform with the associated mathematical rule, and generating three-dimensional implant geometry data configured to guide an additive manufacturing operation.

In yet another embodiment of the present disclosure, a computer system for designing a patient-specific implant includes at least one computer memory that is not a transitory signal, the at least one computer memory including instructions executable by at least one processor for obtaining or uploading computed tomography (CT) data from a spine of a subject, converting the computed tomography (CT) data into a three-dimensional image, selecting or indicating selected segments of the spine for applying surgical correction, applying one or more correction guidelines for the selected segments of the spine, determining whether the selected segments of the spine conform to the one or more correction guidelines, and generating three-dimensional implant geometry data configured to guide an automated manufacturing operation configured to produce an implant.

In still another embodiment of the present disclosure, a patient-specific implant is manufactured by a process including obtaining image data of a region of interest of the spine of a patient, measuring one or more geometric characteristic of the region of interest from the image data, comparing a measurement obtained for at least one of the one or more geometric characteristics to a mathematical rule associated with the particular geometric characteristic, and generating three-dimensional implant geometry data if the measurement of the at least one of the one or more geometric characteristics conforms with the associated mathematical rule, the implant geometry data configured to guide an additive manufacturing operation.

In yet another embodiment of the present disclosure, a patient-specific implant is manufactured by a process including obtaining image data of a region of interest of the spine of a patient, measuring one or more geometric characteristic of the region of interest from the image data, comparing a measurement obtained for each member of a selected group of the one or more geometric characteristics to a mathematical rule associated with the particular geometric characteristic, creating a corrected value for any of the one or more geometric characteristics that do not conform with the associated mathematical rule, and generating three-dimensional implant geometry data configured to guide an additive manufacturing operation.

In still another embodiment of the present disclosure, a patient-specific implant is manufactured by a process including obtaining or uploading computed tomography (CT) data from a spine of a subject, converting the computed tomography (CT) data into a three-dimensional image, selecting or indicating selected segments of the spine for applying surgical correction, applying one or more correction guidelines for the selected segments of the spine, determining whether the selected segments of the spine conform to the one or more correction guidelines, and generating three-dimensional implant geometry data configured to guide an automated manufacturing operation configured to produce an implant.

### BRIEF DESCRIPTION OF THE DRAWINGS

FIGS. 1-22 illustrate a variety of implants configured as intervertebral bodies and spacers, bone plates, pins, dowels, and the like, according to embodiments of the present disclosure.

FIG. 23 illustrates the distal end of an integral implant insertion instrument and implant, according to an embodiment of the present disclosure.

FIG. 24 is a flow chart of a method utilizing a system according to an embodiment of the present disclosure.

FIG. 25 is a side view of a patient with a neutral pelvis.

FIG. 26 is a side view of a patient with anterior pelvic tilt.

FIG. 27 is a side view of a patient with posterior pelvic tilt.

FIG. 28 is a side view of a pelvic tilt angle (PT) in a spine.

FIG. 29 is a side view of C7 sagittal vertebral axis (SVA) in a spine.

FIG. 30 is a side view of lumbar lordosis (LL) and pelvic incidence (PI) in a spine.

FIG. 31 is a side view of an interbody device implanted between two vertebrae of a patient.

FIG. 32 is a plan view of a computer system, according to an embodiment of the present disclosure.

### DETAILED DESCRIPTION

A patient-specific implant and an efficient method of producing the patient-specific implant are described in the embodiments herein. Patient-specific implants according to embodiments described herein may include interbody devices that have been custom-made to not only correspond with the particular anatomy of a patient, including shape and size, but also, to impart a particular type of correction to the patient. The term “interbody device,” as used herein, refers generally to any interbody implant device (interbody device, interbody implant, interbody cage, fusion cage, or spine cage). The interbody devices are typically intended to be placed between two vertebral bodies. Oftentimes, the intervertebral disc is removed prior to the placement of the interbody device. The lower side of an interbody device is intended to abut at least a portion of an upper side (endplate) of a first vertebral body and the upper side of the interbody device is intended to abut at least a portion of a lower side (endplate) of a second vertebral body.

Insufficient contact and load transfer between the vertebral body and the interbody device can produce excessive load transfer in particular locations that can lead the cage to settle or subside into the vertebral body. Furthermore, insufficient contact area or pressure differentials between the interbody device and the vertebral bodies can produce micro-motions and/or macro-motions that can increase subsidence and result in expulsion of the interbody device from the disc space. It is believed by some that this insufficient contact area is due in part to the anatomical variability in the curvature of the vertebral endplates from vertebral level to vertebral level and from patient to patient. Additionally, low bone mineral density index or overaggressive decortications of the endplate can reduce the strength of the endplate and the ability to transfer load from one vertebral body to another. To reduce or eliminate these risks, surgeons carefully prepare the opposing vertebral endplates and attempt to insert an interbody device having as large a footprint (coverage area) as possible, in order to maximize the contact area of the interbody device on the vertebral endplates. When appropriate, the surgeon also places the interbody device on the apophyseal rings to provide as much support and load transfer as possible for spinal distraction, while also ensuring that the interbody device is securely nested within the disc space.

Pre-operative planning software may be utilized to determine or produce implants for surgery. There are two types of pre-operative planning software, those that are device specific and those that are device agnostic. Device specific pre-operative planning software typically provides a method to convert image data, such as CT (computed tomography) scan data, MRI (magnetic resonance induction) data, or x-ray data into two-dimensional or three-dimensional data. The converted data is then used to take measurements of the orthopedic problem (e.g., spine deformity), and to recommend one or more stock or standard model implants for treating the patient. One problem with this strategy is that treatment of the patient is limited to only the device models or sizes that are currently provided by medical device manufacturers. Oftentimes, none of the available models or sizes is appropriate for a particular patient and surgery plan. Complex or large corrections of the spine are typically not possible with the current state of the art in stock implants and device specific pre-operative planning software.

Device agnostic pre-operative planning software is typically used by a medical device manufacturer of a medical professional to provide a custom implant solution for a single patient. Software of this type often provides a method for converting image data (e.g., CT scan, etc.) to three-dimensional data that can be used to measure the orthopedic problem (e.g., spine deformity). Typically, a medical professional works in conjunction with a medical device manufacturer and the applicable regulatory body or controlling organization (FDA, IRB, etc.) to develop a custom device to treat the patient. While this process provides a personalized implant solution, it is an expensive and time-consuming process that is not feasible for most patients, medical professionals, or hospital payer systems.

The systems and methods described herein are configured to provide a three-dimensional shape that represents the ideal implant to fit into the negative space of the spine, once the spine receives the appropriate manipulation in the coronal, sagittal, and axial planes. In other words, the custom shape of the implant will at least partially provide and maintain the desired correction to the spine. Thus, the coronal, sagittal, and axial plane deformities of the spine are corrected, allowing restoration of the anatomical function of the spine. The correction may include both rotation and/or linear displacement along the degrees of freedom. For example, positive displacement along the x-axis, negative displacement along the x-axis, positive rotation around the x-axis, negative rotation around the x-axis, positive displacement along the y-axis, negative displacement along the y-axis, positive rotation around the y-axis, negative rotation around the y-axis, positive displacement along the z-axis, negative displacement along the z-axis, positive rotation around the z-axis, negative rotation around the z-axis. The systems and methods described herein utilize software that is configured to confirm conformity within the validated and regulatory cleared parameters. Upon software verification of the particular design of the interbody device, the software is then configured to capture patient specific data to complete the prescription and transmit the three-dimensional shape and prescription data to the medical device manufacturer. In some embodiments, the hospital or medical facility utilizing the software may itself be the medical device manufacturer. The systems and methods described herein further provide for the manufacture of the implant according to the specifics defined by the software-provided prescription, for example, by additive manufacturing techniques such as three-dimensional printing, or even by subtractive manufacturing techniques, such as CNC-manufacturing. Thus, the turn-around time and the expense, are similar to those for conventional stock implants, although the implants being provided are patient-specific. The patient has the combined benefit of a personalized implant treatment with a conventional price point and a short lead time. There is flexibility, combined with simplicity. The patient's ideal anatomical correction is coded into the prescription and the geometry of the ideal device is sent to a medical device manufacturer for fabrication, or may even be fabricated by a lab in the hospital or other medical treatment site, or remote site.

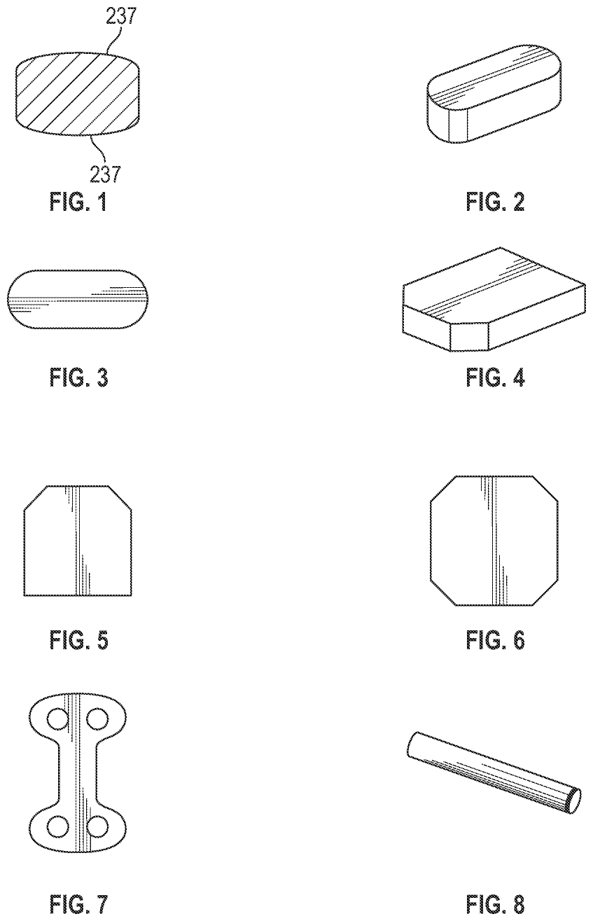

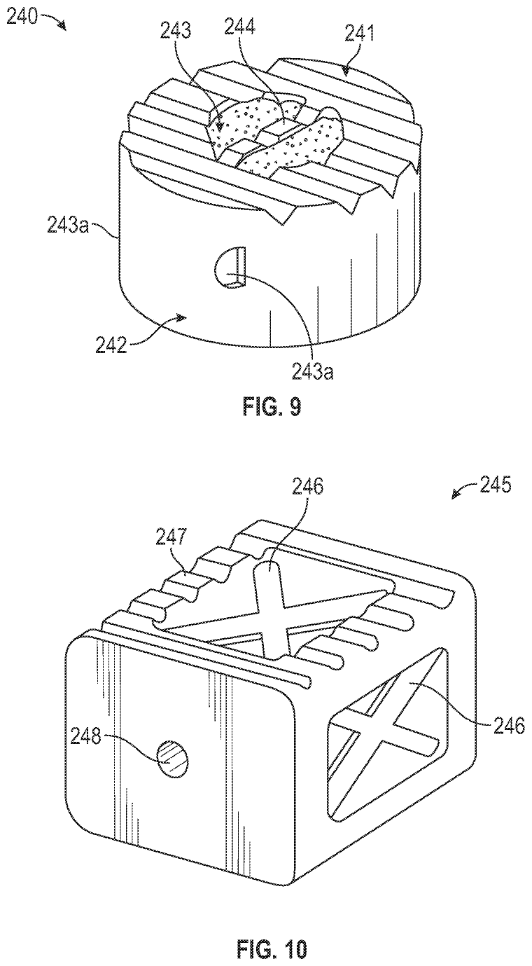

FIGS. 1-22 illustrate a variety of implants which may be produced by the systems and methods described herein, according to several embodiments of the present disclosure. FIG. 1 illustrates an intervertebral implant possessing biconvex surfaces 237 which are configured to match the curvatures of the vertebral endplates with which they come into contact. The biconvex surfaces 237 may each curve laterally (left to right), or curve anteriorly-posteriorly, or curve both laterally and anteriorly-posteriorly (e.g., a hemisphere or other three-dimensional convex shape). FIGS. 2-6 show various views of implants that are suitable for interbody use, including the oval footprints of FIGS. 2-3, the hexagonal footprints of FIGS. 4-5, or the octagonal footprint of FIG. 6. In some embodiments, other polygonal footprints may be utilized. In some embodiments, the footprint may comprise sides that are equal in length to each other, or in other embodiments, all of the sides may be equal in length to each other, or in other embodiments, none of the sides may be equal in length to each other. FIG. 7 shows a cervical bone plate for the fixation of adjacent cervical verterbrae. The bone plate spans two or more vertrebrae, but does not necessarily fill the space between two vertebral endplates. The size and shape of the bone plate of FIG. 7, including the location of holes for screws, may be configured to maintain the cervical vertebrae in a particular relation to each other. FIG. 8 illustrates a cylindrical pin or dowel which may be keyed (not shown) to facilitate its installation. FIG. 9 illustrates an intervertebral implant 240 having a textured surface 241, e.g., roughenings, knurlings, ridges, and the like, to resist backing-out of the implant following its insertion in the intervertebral space. Surfaces 241 may converge to provide an anterior ramp configuration possessing a suitable lordotic angle or the surfaces may be essentially flat. The outer profile 242 of the implant can be round, oval, square, diamond-shaped, octagonal, hexagonal, etc., as requirements suggest. The implant can be provided with an opening 243 for receiving a quantity of osteogenic/osteoinductive material and/or a rigid reinforcing member 244 for added strength. The walls of the implant possess a pair of inserter instrument interfaces 243a (only one shown) for engagement with one end of an insertion tool. FIG. 10 depicts an open, or cage-like, structure 245 suitable for use as an anterior or posterior intervertebral implant. Cross braces 246 on one or more sides of the implant provide increased structural strength over that of a totally open configuration. The open space can be advantageously filled with an osteogenic/osteoinducting material. Texturized surfaces, e.g., ridges, 247 are provided to resist backing-out of the implant following its installation. Inserter interface 248 is intended to receive the distal (working end) of an implant insertion tool. FIG. 11 illustrates an intervertebral implant 249 possessing a position-retaining textured surface 250 (ridges) and a pattern of orifices communicating with the interior which possesses a void structure. The sides of the implant at one end thereof have a matching pair of implant inserter interfaces 252 which are intended to be grasped by an insertion tool. One end of the implant possesses an orifice 249a through which an osteogenic/osteoinductive material can be introduced into the interior void communicating channels of the implant.

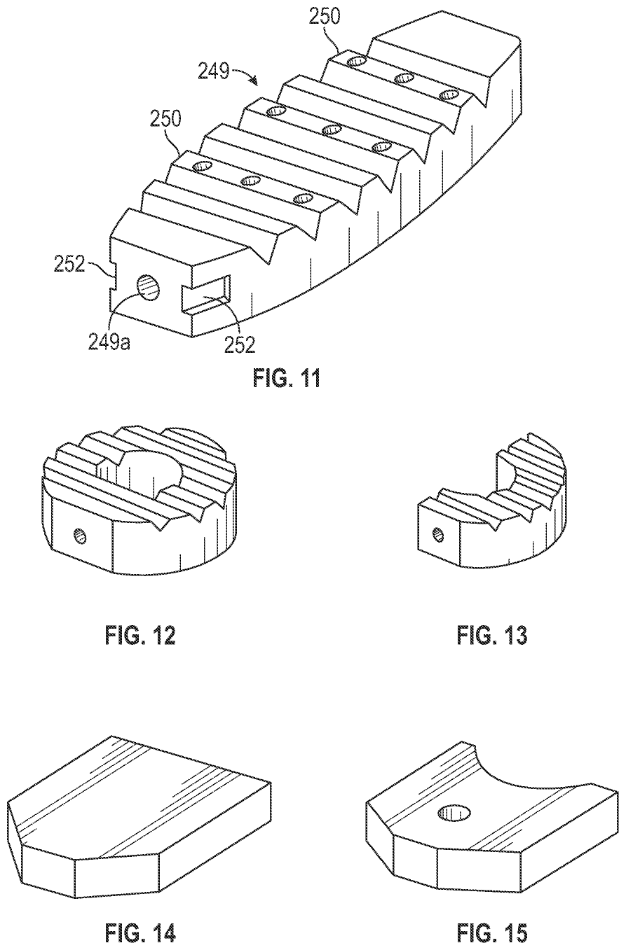

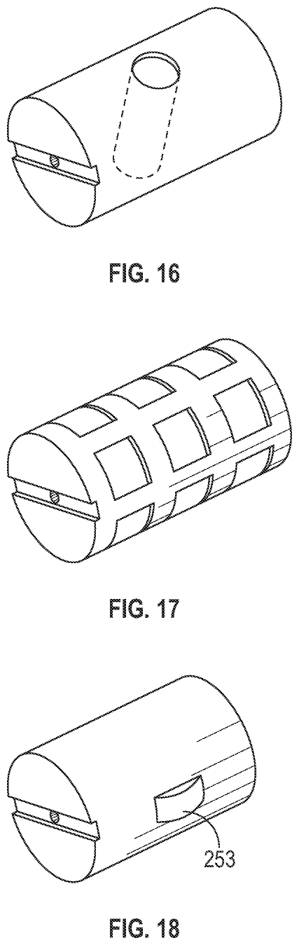

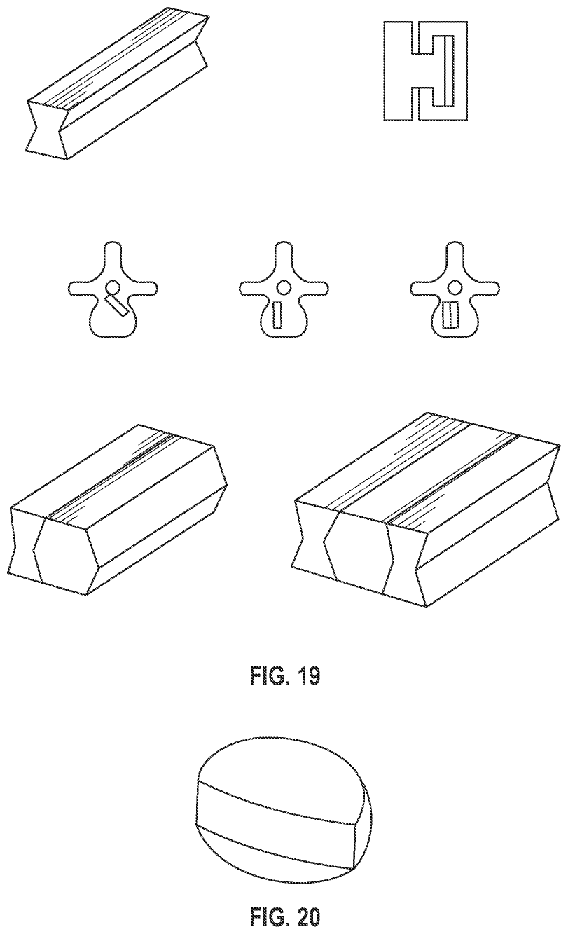

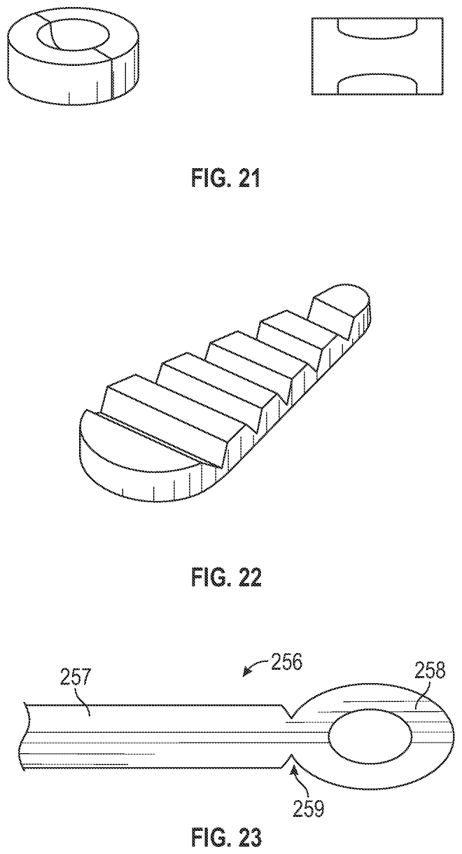

FIGS. 12-15 show various additional configurations of implants for insertion in the intervertebral space: FIG. 12 (an anterior ramp implant), FIG. 13 (a posterior ramp implant), FIG. 14 (a cervical spacer) and FIG. 15 (a cervical spacer including a radiused recess at one side). FIGS. 16-18 show various implants configured as intervertebral dowels. The implant of FIG. 16 is a solid structure with a through bore for receiving osteogenic/osteoinductive material. The implant of FIG. 17 possesses an open, or cage-like, structure which can be packed with osteogenic/osteoinductive material. The implant of FIG. 18 possesses at least one wing-like structure 253 on its longitudinal surface which prevents rotational displacement within the intervertebral space. Each of implants 16-18 possesses a slot and central hole for receiving the distal end of an insertion tool. FIG. 19 illustrates a transforaminal lumbar interbody fusion (TLIF) implant and its assembly from subunits. The implant of FIG. 19 may alternatively be used as a posterior lumbar interbody fusion (PLIF) implant. FIG. 20 depicts a convex anterior interbody ramp with openings communicating with the interior. FIG. 21 shows another embodiment of anterior interbody implant. FIG. 22 shows a solid anterior interbody implant presenting a large surface area for implant-vertebral endplate contact.

FIG. 23 schematically illustrates in plan view the distal end 257 of an integral, or combined, implant insertion instrument and implant 256 wherein implant portion 258 specifically, an intervertebral implant, is joined to the distal end 257 of the instrument portion through a weakened, or break-away, site of attachment 259. Following insertion of the intervertebral implant in the intervertebral space, application of a sharp upward or downward movement of the implant insertion instrument will result in the distal end of the instrument cleanly breaking away, and separating from, the implant which remains in place. Alternatively, the site of attachment 259 may comprise any mechanism that allows the distal end 257 to releasably grip or maintain the implant 258, including, but not limited to: a clamp, an adhesive, epoxy, or hot melt attachment, a magnetic connection, a snap, a threaded attachment, a tapered attachment, a spring attachment, and a combination of any two or more of these attachment features. Custom instruments may even be produced by the systems and methods described herein. For example, a particular patient may have a deformity which requires an instrument having a particular angle that is not available in off-the-shelf instruments.

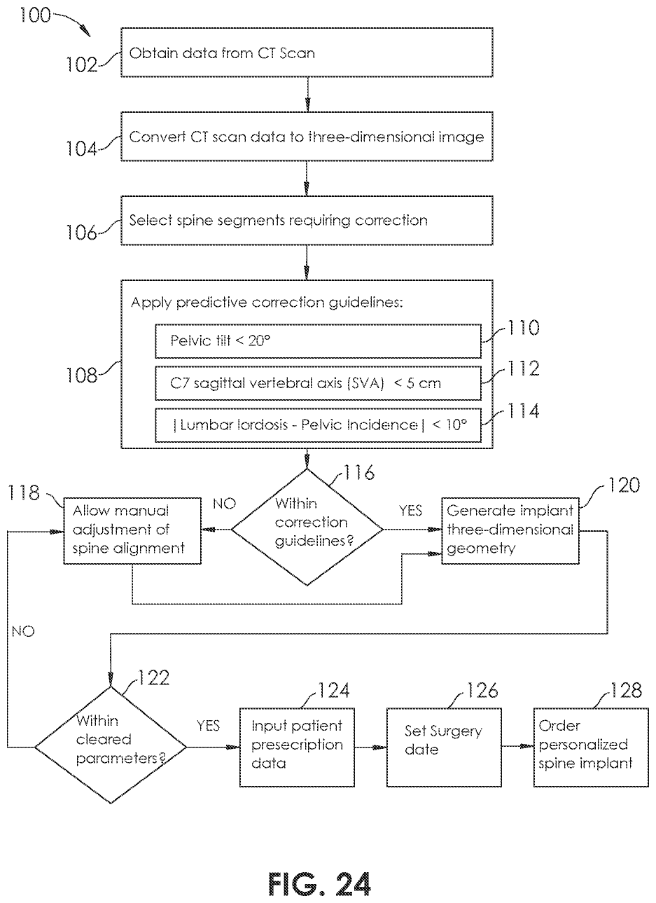

FIG. 24 illustrates a method 100 for utilizing a system for producing patient-specific implants. In step 102 scan data is obtained from a CT scan of a patient, for example, a CT scan that includes the spine of the patient, or at least the portion of interest in the spine. In other embodiments the scan data may comprise MRI scan data or x-ray data. In step 104, the CT scan data is converted into a three-dimensional image through software manipulation of the data. Typically, CT scan data is presented in a DICOM format, which includes individual slices of imaging data. A common slide thickness is one mm, though other thickness may be used, such as 0.25 mm, 0.5 mm, 1.5 mm, 2.0 mm, 2.5 mm, 3.0 mm and greater thicknesses. In step 106 spine segments may be selected for analysis. For example, a user interface that is associated with at least one computer memory that is not a transitory signal and which comprises instructions executable by at least one processor may be utilized to select a region of interest. In some cases, the region of interest may be a diseased or deformed portion of the spine, including a particular number of successive vertebrae and their surrounding soft tissue. In some cases, the entire spine may be selected. In some cases, only sacral and lumbar vertebrae and their surrounding soft tissue are selected. In some cases, only lumbar and thoracic vertebrae and their surrounding soft tissue are selected. In some cases, only cervical vertebrae and their surrounding soft tissue are selected.

Turning to FIG. 32, a system 330 containing a memory 332 and a processor 334 may include any number of custom stand-alone devices, or any mobile device, such as an iPhone, smart phone, iPAD, smart watch, laptop or desktop computer. The system 330 may also include a user interface 336. The system 330 may also be configured to access the memory 332 remotely, for example, via internet browser access or other wireless means 338. The three-dimensional image can be converted into a form such that it can be manipulated by a user to measure anatomical deformities related to the disease (e.g., spine disease). The information can then be used by a medical professional, or technical or engineering professional in conjunction or collaboration with a medical professional, to design the optimized geometry of the corrected spine, thus allowing the design of an implant to treat the particular disease or malady.

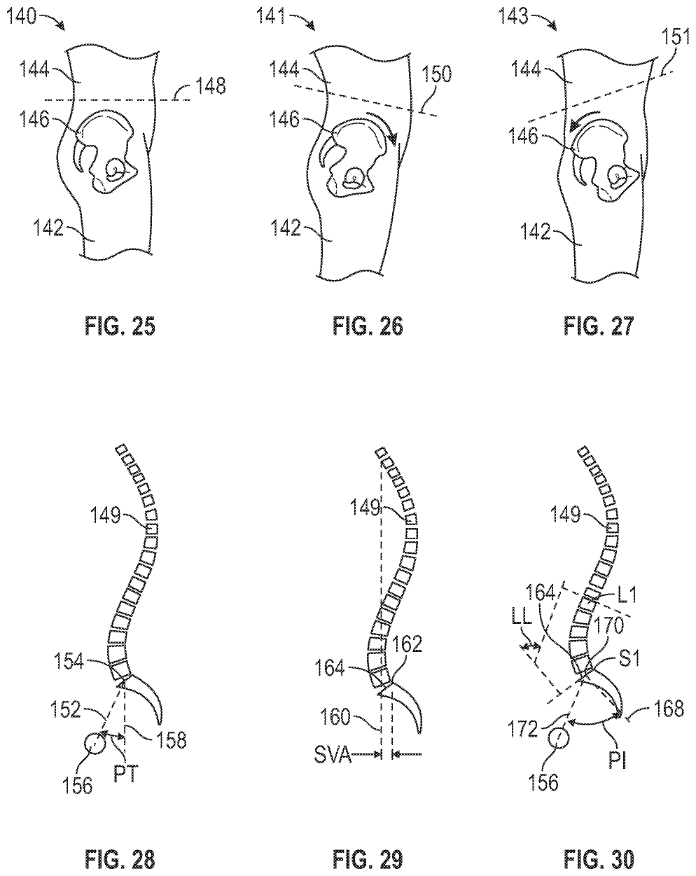

In step 108, the computer memory is utilized to apply one or more predictive correction guidelines to the spine or to the selected portion of the spine, or at least a section thereof. A number of predictive correction guidelines may be utilized, but in one embodiment a set of three predictive guidelines is applied, relating to pelvic tilt 110, sagittal alignment 112, and lumbar lordosis 114. Legs 142 and torso 144 of a standing patient 140 are shown in FIG. 25. The patient 140 has a pelvis 146 that is in a neutral condition in relation to horizontal line 148. FIG. 26 illustrates a standing patient 141 who has an anterior pelvic tilt, with forwardly rotated pelvis 146 indicated by curved arrow and line 150 that is angled in relation to horizontal line 148. FIG. 27 illustrates a standing patient 143 who has a posterior pelvic tilt, with backwardly rotated pelvis 146 indicated by curved arrow and line 151 that is angled in relation to horizontal line 148. A spine 149 of a patient is shown in FIG. 28. A pelvic tilt angle PT is the angle created by the vertical axis 158 and a line 152 running from a midpoint of the sacral end plate 154 to the center 156 of the bifemoral heads. The predictive guideline regarding pelvic tilt 110 is defined by the equation wherein the pelvic tilt angle PT is less than 20 degrees (PT<20°). In some embodiments, the predictive guideline regarding pelvic tilt 110 may be defined by the equation 0°<PT<20°.

FIG. 29 illustrates a spine 149 having a positive C7 sagittal vertebral axis SVA. The C7 sagittal vertebral axis SVA is the distance from the plumb line 160 dropping from the center of the C7 vertebra to the posterior edge 162 of the upper sacral endplate surface 164. The predictive guideline regarding C7 sagittal vertebral axis (SVA) 112 is defined by the equation wherein the a C7 sagittal vertebral axis (SVA) less than 5 cm (SVA<5 cm). In some embodiments, the predictive guideline regarding the C7 sagittal vertebral axis (SVA) is defined by the equation wherein the absolute value of the C7 sagittal vertebral axis (SVA) is less than 5 cm (|SVA|<5 cm).

FIG. 30 illustrates a spine 149 with pelvic incidence PI and lumbar lordosis LL indicated. The pelvic incidence PI is the degree of sacral tilt with respect to the pelvis. More specifically, the pelvic incidence PI is the angle between the perpendicular line 168 to the center point 170 of the upper sacral endplate surface 164 and the line 172 between the center point 170 and center 156 of the bifemoral heads. Lumbar lordosis LL is the angle between L1 and S1. The predictive guideline regarding lumbar lordosis 114 is defined by the equation wherein an absolute value of the difference between pelvic incidence PI and lumbar lordosis LL is less than about 10 degrees. More specifically, the predictive guideline regarding lumbar lordosis 114 may be defined by the equation wherein an absolute value of the difference between pelvic incidence PI and lumbar lordosis LL is less than about 9 degrees. In some embodiments, other parameters may be used to formulate alternative or additional corrective guidelines. For example: C2-C7 angle, which evaluates the degree of cervical lordosis, and is the angle between the upper endplate surface of the C2 vertebra and the lower endplate surface of the C7 vertebra; thoracic kyphosis T1-T12 angle, which is the angle between the upper endplate surface of the T1 vertebra and the lower endplate surface of the T12 vertebra; thoracic kyphosis T4-T12 angle, which is the angle between the upper endplate surface of the T4 vertebra and the lower endplate surface of the T12 vertebra; C7 slope angle, which may predict cervical lordosis and thoracic kyphosis, and which is the angle between a horizontal line and the upper endplate surface of the C7 vertebra; sacral slope, which is the angle between a horizontal lune and the slope of the upper sacral endplate surface. Sacral slope SS may have a correlation between with pelvic incidence PI and pelvic tilt PT, such that SS≈PI−PT.

In decision point 116, the computer memory is utilized to determine whether, in the current state of the spine provided by the three-dimensional image, the predictive guidelines 110, 112, 114 are achieved. If one or more of the predictive guidelines 110, 112, 114 are not true for the spine segments selected, then a user may utilize a user interface to adjust spine alignment, as shown in step 118. For example, if the pelvic tilt is determined to be 20° or greater, a user may input or toggle an adjustment that changes the amount of correction in order to achieve a pelvic tilt less than 20°. If it is determined that the predictive guidelines are all achieved (whether user adjustment was or was not required), the system generates three-dimensional geometry in step 120. The three-dimensional geometry may in some cases define a single interbody device, or in other cases may define several interbody devices. In some cases, the three-dimensional geometry may define one or more interbody devices for a single level of the spine, or in other cases may define one or more interbody devices from two or more levels of the spine. In one embodiment, the DICOM data creates a point cloud map, which is then converted to multiple interconnected triangles to create a surface mesh. Based on known density discrepancies between bone and tissue, the three-dimensional mesh surface is parsed for bone surface data and converted to a three-dimensional image with volume. The converted data is saved into memory with a readable file format, such as .STL, .OBJ, or other CAD (computer-aided design) readable file format. In this CAD readable file format, the individual spine vertebral bodies can be isolated and manipulated in the axial, coronal, and sagittal planes.

After the three-dimensional geometry is generated, the system checks in decision point 122 whether the particular correction is within cleared parameters. For example, within a particular amount of correction that is approved under a regulatory clearance; or, within a particular amount of correction that is approved under an IRB-controlled or FDA-controlled clinical trial. In addition to, or instead of, the amount of correction, other parameters may determine whether the three-dimensional geometry performs within cleared parameters in decision point 122. For example, the total volume or total mass of the implant(s) may be controlled, such that it must be within a particular range, or that it must be below a certain amount per unit weight of patient. If the correction (or other parameters) is not within the cleared range(s), user-initiated input may be performed, as in step 118. In some embodiments, the system may suggest the amount to adjust each parameter of spine alignment, allowing the user to accept this suggestion, or to choose a different value of change. In some cases, step 122 may not be necessary, for example, when certain procedures do not have implant-based regulatory limitations. A particular manner of validating a cleared amount of correction, is to check the three-dimensional envelope of the spine implant at both the maximum material condition and the least material condition. For example, an FDA clearance may take into account both of these conditions, in one or more patient indications.

Once the three-dimensional geometry is accepted by the user, and, if applicable, by the limitations of step 122, a patient prescription can be created in step 124. The patient prescription may comprise one or more three-dimensional files that are used in additive manufacturing, including, but not limited to: .AMF, .X3D, Collada (Collaborative Design Activity), .STL, .STP, .STEP, or .OBJ. The patient prescription may alternatively comprise one or more three dimensional files, including, but not limited to: .IGS, .STP, .STEP, .3ds, .blend, .dae, .ipt, .skp, .fbx, .lwo, .off, .ply, .sldprt, .sldasm, and .X_T. In some cases, the patient prescription may also include one or more two-dimensional files, for example, to map or guide the surgical treatment, or to stage the utilization of each implant. The two-dimensional files may include, but are not limited to: .dwg, .dwf, .dxf, .pdf, or .acis. The surgery can be scheduled in step 126, and the personalized implant can be ordered in step 128.

The step 128 may include using the three-dimensional files to manufacture the implant using one or more additive manufacturing or subtractive manufacturing methods. Additive manufacturing methods include, but are not limited to: three-dimensional printing, stereolithography (SLA), selective laser melting (SLM), powder bed printing (PP), selective laser sintering (SLS), selective heat sintering (SHM), fused deposition modeling (FDM), direct metal laser sintering (DMLS), laminated object manufacturing (LOM), thermoplastic printing, direct material deposition (DMD), digital light processing (DLP), inkjet photo resin machining, and electron beam melting (EBM). Subtractive manufacturing methods include, but are not limited to: CNC machining, EDM (electrical discharge machining), grinding, laser cutting, water jet machining, and manual machining (milling, lathe/turning).

Following the manufacture of the implant, a bone-friendly scaffold is created for fusion to one or more vertebrae. The implant may comprise one or more of the following materials: titanium, titanium alloy, titanium-6AL-4V, tantalum, and PEEK (polyether ether ketone). The implant may also comprise/be coated with a biologic material. Examples of potential biological materials include, but are not limited to: hydroxylapetite (hydroxyapetite), recombinant human bone morphogenic proteins (rhBMP-2, rhBMP-7), bioactive glass, beta tri-calcium phosphate, human allograft (cortical and/or cancellous bone), xenograft, other allograft, platelet rich plasma (PRP), stem cells, and other biomaterials. In addition, synthetic ceramics having osteogenic properties may be utilized. The manufacture of the implant may be further guided by patient information, including patient age, patient weight, or prior patient surgical history. For example, a patient with a high BMI (body mass index) can require a stiffer or stronger implant, such as an implant made with a different material (e.g., having mechanical characteristics) and/or an implant having thicker material cross-sectional dimensions. The lattice structure forming the implant can be optimized to meet the patient's biomechanical needs for stability. Additionally, a patient with a low BMI and/or with osteoporotic bone or osteopenia can benefit from an implant having lower stiffness (higher flexibility), thus helping to reduce the risk of subsidence. Furthermore, a patient having a previously failed fusion may be at risk for adjacent level disc disease and/or proximal joint kyphosis. An implant can be tailored to alleviate this particular situation.

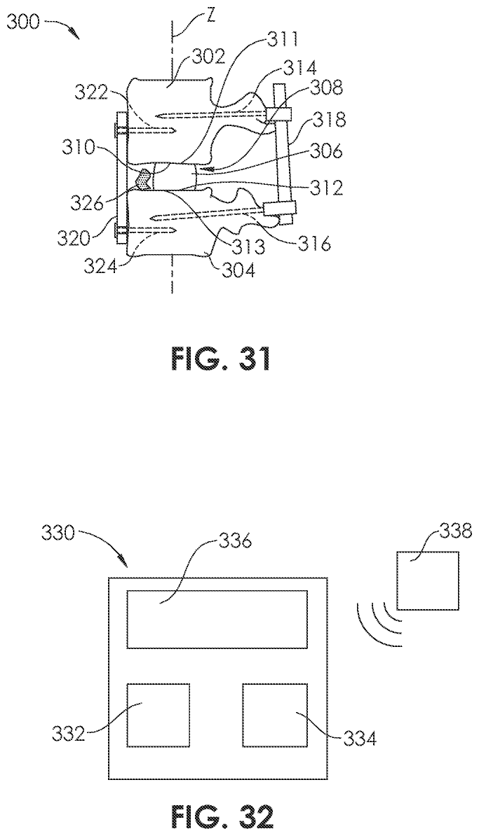

FIG. 31 illustrates a portion of a spine 300 including a first vertebra 302 and a second vertebra 304. In use, a patient-specific interbody device 306 manufactured using according to the embodiments described herein is placed by a user into the negative space 308 of the spine 300, and engaged between a lower endplate surface 310 of the first vertebra 302 and an upper endplate surface 312 of the second vertebra 304. An upper surface 311 of the interbody device 306 engages the lower endplate surface 310 of the first vertebra 302 and a lower surface 313 of the interbody device 306 engages the upper endplate surface 312 of the second vertebra 304. The interbody device 306 is configured to maintain the distance between the first vertebra 302 and the second vertebra 304, and more specifically, between the lower endplate surface 310 of the first vertebra 302 and the upper endplate surface 312 of the second vertebra 304 (e.g., along the Z-axis). The interbody device 306 may also be configured to maintain X-axis and/or Y-axis position (not shown) between the first vertebra 302 and the second vertebra 304, and may be configured to maintain rotational orientation of one of both of the first vertebra 302 and the second vertebra 304, for example, in relation to the X-axis, Y-axis, and/or Z-axis. In some embodiments, pedicle screws 314, 316 and a rod 318 may be used to further lock the orientation in place. In other embodiments, a plate 320 and screws 322, 324 may be used to further lock the orientation in place. Though both the rod 318 and plate 320 are shown coupled to the first vertebra 302 and the second vertebra 304 in FIG. 31, commonly only one of two would be used, though it is possible to incorporate both in certain situations. Bone graft 326 or other biological materials may be used to further promote fusion of the first vertebra 302 and the second vertebra 304, and may be implanted within the negative space 308.

The systems and methods described herein may be utilized to correct other physiological ailments requiring a patient-specific implant. For example, wedge-shaped implants for maintaining wedge osteotomies in the spine, or other orthopedic areas such as the hip, jaw, chin or knee for arthritic or non-arthritic conditions, may be designed with the teachings of the present disclosure. Particular procedures include: high tibial osteotomy (tibia), distal femoral osteotomy (femur), Evans wedge or Cotton wedge (foot and ankle).

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately,” “about,” and “substantially” as used herein include the recited numbers (e.g., about 10%=10%), and also represent an amount close to the stated amount that still performs a desired function or achieves a desired result. For example, the terms “approximately,” “about,” and “substantially” may refer to an amount that is within less than 10% of, within less than 5% of, within less than 1% of, within less than 0.1% of, and within less than 0.01% of the stated amount.

While embodiments have been shown and described, various modifications may be made without departing from the scope of the inventive concepts disclosed herein.

1-66. (canceled)

67. A computer-implemented method for designing a patient-specific implant, comprising:
measuring, from a model of patient spinal anatomy that includes a proposed surgical correction to the patient spinal anatomy, one or more geometric characteristics of the patient spinal anatomy associated with the proposed surgical correction;
comparing a measured value of at least one of the one or more geometric characteristics to one or more criteria associated with the at least one geometric characteristic;
in response to the measured value meeting the one or more criteria based on the comparison, generating implant geometry data based at least in part on the model and/or the measured value, wherein the implant geometry data includes a three-dimensional geometry of a patient-specific implant;
in response to the measured value not meeting the one or more criteria based on the comparison, requesting an adjustment to the proposed surgical correction to patient spinal anatomy such that the at least one geometric characteristic meets the one or more criteria before generating the implant geometry data; and
causing a manufacturing system to manufacture the patient-specific implant based on the implant geometry data.

68. The computer-implemented method of claim 67, further comprising:
generating the model of the patient spinal anatomy that includes the proposed surgical correction,
wherein generating the model includes receiving, from a user, one or more inputs at least partially defining the proposed surgical correction.

69. The computer-implemented method of claim 67 wherein the model is a three-dimensional virtual model.

70. The computer-implemented method of claim 67 wherein the model is adjustable.

71. The computer-implemented method of claim 67 wherein the one or more geometric characteristics include pelvic tilt, sagittal alignment, and/or lumbar lordosis.

72. The computer-implemented method of claim 71 wherein the one or more criteria includes a predetermined range of values and/or a predetermined threshold for one or more of:
pelvic tilt,
sagittal alignment, and/or
lumbar lordosis.

73. The computer-implemented method of claim 67 wherein the one or more geometric characteristics include sagittal vertical axis, lumbar vertical axis, coronal offset, cobb angle, and/or coronal angle.

74. The computer-implemented method of claim 67 wherein the patient-specific implant includes an intervertebral implant having a surface configured to match a surface of a vertebral endplate the patient-specific implant is designed to be implanted at.

75. The computer-implemented method of claim 67 wherein the patient-specific implant includes a cervical plate.

76. The computer-implemented method of claim 67 wherein the patient-specific implant includes a rod.

77. A method for designing a patient-specific implant, comprising:
measuring, from a model of patient spinal anatomy that includes a proposed surgical correction to the patient spinal anatomy, one or more geometric characteristics of the patient spinal anatomy associated with the proposed surgical correction;
determining whether a measurement obtained for at least one of the one or more geometric characteristics of the patient spinal anatomy meets one or more criteria associated with the at least one geometric characteristic;
generating three-dimensional implant geometry data based at least in part on the model and in response to the measurement of the at least one of the one or more geometric characteristics meeting the one or more criteria; and
causing a manufacturing system to manufacture the patient-specific implant based on the implant geometry data.

78. The method of claim 77 wherein the one or more geometric characteristics include pelvic tilt, sagittal alignment, lumbar lordosis, sagittal vertical axis, lumbar vertical axis, coronal offset, cobb angle, and/or coronal angle.

79. The method of claim 78 wherein the one or more criteria includes a predetermined range of values and/or a predetermined threshold for one or more of pelvic tilt, sagittal alignment, lumbar lordosis, sagittal vertical axis, lumbar vertical axis, coronal offset, cobb angle, and/or coronal angle.

80. A non-transitory computer-readable medium storing instructions that, when executed by a computing system, cause the computing system to perform operations comprising:
measuring, from a model of patient spinal anatomy that includes a proposed surgical correction to the patient spinal anatomy, one or more geometric characteristics of the patient spinal anatomy associated with the proposed surgical correction;
comparing a measured value of at least one of the one or more geometric characteristics to one or more criteria associated with the at least one geometric characteristic;
in response to the measured value meeting the one or more criteria based on the comparison, generating implant geometry data based at least in part on the model and/or the measured value, wherein the implant geometry data includes a three-dimensional geometry of a patient-specific implant;
in response to the measured value not meeting the one or more criteria based on the comparison, requesting an adjustment to the proposed surgical correction to patient spinal anatomy such that the at least one geometric characteristic meets the one or more criteria before generating the implant geometry data; and
causing a manufacturing system to manufacture the patient-specific implant based on the implant geometry data.

81. The non-transitory computer-readable medium of claim 80 wherein the operations further comprise:
generating the model of the patient spinal anatomy that includes the proposed surgical correction,
wherein generating the model includes receiving, from a user, one or more inputs at least partially defining the proposed surgical correction.

82. The non-transitory computer-readable medium of claim 80 wherein the model is an adjustable three-dimensional virtual model.

83. The non-transitory computer-readable medium of claim 80 wherein the one or more geometric characteristics include pelvic tilt, sagittal alignment, lumbar lordosis, sagittal vertical axis, lumbar vertical axis, coronal offset, cobb angle, and/or coronal angle.

84. The non-transitory computer-readable medium of claim 83 wherein the one or more criteria includes a predetermined range of values and/or a predetermined threshold for one or more of pelvic tilt, sagittal alignment, lumbar lordosis, sagittal vertical axis, lumbar vertical axis, coronal offset, cobb angle, and/or coronal angle.

85. The non-transitory computer-readable medium of claim 80 wherein the patient-specific implant includes a cage.

86. The non-transitory computer-readable medium of claim 80 wherein the patient-specific implant includes a plate and/or a rod.

87. A system for designing a patient-specific implant, comprising:
at least one processor;
at least one non-transitory computer memory comprising instructions executable by the at least one processor for:
measuring, from a model of patient spinal anatomy that includes a proposed surgical correction to the patient spinal anatomy, one or more geometric characteristics of the patient spinal anatomy associated with the proposed surgical correction,
determining whether a measurement obtained for at least one of the one or more geometric characteristics of the patient spinal anatomy meets one or more criteria associated with the at least one geometric characteristic, and
generating three-dimensional implant geometry data based at least in part on the model and in response to the measurement of the at least one of the one or more geometric characteristics meeting the one or more criteria; and
a manufacturing system configured to manufacture the patient-specific implant using the implant geometry data.

88. The system of claim 87 wherein the operations further comprise:
generating the model of the patient spinal anatomy that includes the proposed surgical correction,
wherein generating the model includes receiving, from a user, one or more inputs at least partially defining the proposed surgical correction.

89. The system of claim 87 wherein the one or more geometric characteristics include pelvic tilt, sagittal alignment, lumbar lordosis, sagittal vertical axis, lumbar vertical axis, coronal offset, cobb angle, and/or coronal angle.

90. The system of claim 89 wherein the one or more criteria includes a predetermined range of values and/or a predetermined threshold for one or more of pelvic tilt, sagittal alignment, lumbar lordosis, sagittal vertical axis, lumbar vertical axis, coronal offset, cobb angle, and/or coronal angle.

91. The system of claim 87 wherein the patient-specific implant includes a cage.
